(19) United States
(12) Patent Application Publication
(10) Pub. No.: US 2026/0341500 A1
(43) Pub. Date: Oct. 29, 2026

(54) SYSTEMS AND METHODS CONFIGURED FOR ENHANCING IMAGING INTERPRETATIONS

(71) Applicant: THE FEINSTEIN INSTITUTES FOR MEDICAL RESEARCH, INC., Manhasset, NY (US)

(72) Inventors: James THANNICKAL, Manhasset, NY (US); Matthew BARISH, Manhasset, NY (US); Daniel NICOSIA, Manhasset, NY (US); David HIRSCHORN, Manhasset, NY (US)

(73) Assignee: THE FEINSTEIN INSTITUTES FOR MEDICAL RESEARCH, INC., Manhasset, NY (US)

(21) Appl. No.: 19/653,654

(22) Filed: Apr. 21, 2026

Related U.S. Application Data
(60) Provisional application No. 63/793,107, filed on Apr. 23, 2025.
(60) Provisional application No. 63/855,522, filed on Aug. 1, 2025.

(51) Int. Cl. G16H 40/67; G06V 10/776; G06V 10/778; G16H 15/00; G16H 30/20

(52) U.S. Cl. CPC G16H 40/67; G06V 10/776; G06V 10/7788; G16H 15/00; G16H 30/20; G06V 2201/03

(57) ABSTRACT

A method includes transmitting a medical image of a subject case to a remote computer with an machine-learning (ML) model for processing the medical image, displaying the medical image at a local workstation, monitoring a predetermined status indication at the local workstation, the predetermined status indication being associated with the medical image, displaying at least one model output corresponding to the medical image from the remote computer upon the local workstation detecting a change in the predetermined status indication, capturing at least one interaction with the at least one model output at the local workstation, and transmitting the at least one interaction with an identification pointing to the medical image to the remote computer.

### CROSS REFERENCE TO RELATED APPLICATION

This application claims the benefit or priority to U.S. Provisional Application No. 63/793,107, filed Apr. 23, 2025, and U.S. Provisional Application No. 63/855,522, filed Aug. 1, 2025, the specifications and drawings of which are incorporated herein by reference.

### FIELD OF TECHNOLOGY

The present disclosure generally relates to the field of imaging interpretations.

### BACKGROUND OF TECHNOLOGY

Radiology, also called medical imaging, is employed in a wide range of medical fields for diagnosis and monitoring of diseases and other medical conditions. Examples of radiological imaging modalities include magnetic resonance imaging (MRI), computed tomography (CT) and other X-ray imaging modalities, emission imaging modalities such as positron emission tomography (PET) and single photon emission computed tomography (SPECT), and so forth. Typically, radiological imaging can also be classified by medical field, e.g., oncology imaging, sports medicine imaging, cardiac imaging, image guided therapy (iGT), and so forth. The radiology workflow may be generally divided into several phases, including the image acquisition, image interpretation, and clinical application phases. Image acquisition is typically performed by an imaging technician or technologist or operator or the like. Image interpretation is typically performed by a radiologist, who is typically a medical doctor (e.g., an M.D.) specializing in interpretation of radiology image studies. Clinical application is typically performed by the subject's doctor, e.g., a general practitioner doctor or specialist such as an oncologist, cardiologist, orthopedic surgeon, or the like.

### SUMMARY OF DESCRIBED SUBJECT MATTER

The present disclosure is directed to exemplary computer systems, computing devices, and software for enhancing the accuracy and efficiency in medical imaging interpretation workflows. Machine-learning (ML), e.g., image recognition is deployed at a right time during a medical imaging interpretation workflow to improve the quality of the medical image interpretation and/or the quality of the AI inferencing.

In at least some embodiments, the techniques described herein relate to a computer-implemented method including: transmitting, by at least one computing device, a first medical image of a first subject case to a first remote computer with a first machine-learning (ML) model for processing the first medical image; displaying, by the at least one computing device, the first medical image at a local workstation; monitoring, by the at least one computing device, a predetermined status indication at the local workstation, the predetermined status indication being associated with the first medical image; displaying, by the at least one computing device, at least one first model output corresponding to the first medical image from the first remote computer upon the local workstation detecting a change in the predetermined status indication; capturing, by the at least one computing device, at least one first interaction with the at least one first model output at the local workstation; and transmitting, by the at least one computing device, the at least one first interaction with an identification pointing to the first medical image to the first remote computer.

In at least some embodiments, the techniques described herein relate to a method, further including displaying, by the at least one computing device, a text box at the local workstation for entering a text, wherein the change in the status indication is triggered by a number of characters entered in the text box exceeding a predetermined threshold.

In at least some embodiments, the techniques described herein relate to a method, further including displaying, by the at least one computing device, a button at the local workstation, wherein a clicking of the button triggers a change of the status indication.

In at least some embodiments, the techniques described herein relate to a method, wherein the button switches from invisible to visible upon a text curser being placed in the text box.

In at least some embodiments, the techniques described herein relate to a method, wherein the change in the predetermined status indication corresponds to completion of a diagnostic conclusion section of a radiology report.

In at least some embodiments, the techniques described herein relate to a method, further including: transmitting, by the at least one computing device, a second medical image of a second subject case to the first remote computer with the first machine-learning model for processing the second medical image; receiving, by the at least one computing device, an indication that the second subject case has lower priority ranking than the first subject case before displaying the first medical image at the local workstation.

In at least some embodiments, the techniques described herein relate to a method, further including incrementing, by the at least one computing device, a counter by 1 in response to transmitting the at least one first interaction to the first remote computer.

In at least some embodiments, the techniques described herein relate to a method, wherein the first remote computer retrains the first ML model with the captured at least one first interaction.

In at least some embodiments, the techniques described herein relate to a method, further including: transmitting, by the at least one computing device, the first medical image to a second remote computer with a second ML model for processing the first medical image; receiving, by the at least one computing device, at least one second model output corresponding to the first medical image from the second remote computer; and displaying, by the at least one computing device, the at least one second model output along with the at least one first model output.

In at least some embodiments, the techniques described herein relate to a method, further including normalizing the at least one first and second model output into a predetermined schema including at least one of: standardized labels, confidence scores, anatomical locations, or clinical categories.

In at least some embodiments, the techniques described herein relate to a method, further including comparing the at least one first model output with the at least one second model output to identify agreement, discrepancy, or conflict therebetween.

In at least some embodiments, the techniques described herein relate to a method, further including displaying a user-selectable control that allows a radiologist to manually retrieve the at least one first model output after the local workstation has detected the change in the predetermined status indication.

In at least some embodiments, the techniques described herein relate to a method, wherein at least one first interaction includes an accept, reject, or modify action applied to the at least one first model output.

In at least some embodiments, the techniques described herein relate to a method, further including anonymizing, by the computing device, the at least one first interaction before transmitting the at least one first interaction to the first remote computer.

In at least some embodiments, the techniques described herein relate to a method, further including providing, by the at least one computing device, an integrated procedure complication reporting interface configured to capture structured data identifying a procedural complication type, a complication category, and associated descriptive information during or after interpretation of the first medical image.

In at least some embodiments, the techniques described herein relate to a method, wherein the integrated procedure complication reporting interface includes a validation logic adapted to prevent submission of a complication report unless a predetermined field has been completed.

In at least some embodiments, the techniques described herein relate to a method, further including automatically categorizing, by the at least one computing device, the at least one first model output within a clinical report into a predefined category including at least one of critical, urgent, or incidental findings, and updating a navigation interface based on the categorization, wherein the navigation interface is configured to allow a user to selectively access categorized findings and corresponding follow-up actions without manual tracking.

In at least some embodiments, the techniques described herein relate to a method, further including detecting, by the at least one computing device, a duplicate subject identifier associated with the first medical image and presenting, by the at least one computing device, at least one prior imaging study associated with the duplicate identifier at the local workstation.

In at least some embodiments, the techniques described herein relate to a non-transitory computer readable medium storing program instructions that, when executed by a processor, cause a computer system to perform operations including: transmitting a first medical image of a first subject case to a first remote computer with a first machine-learning (ML) model for processing the first medical image; displaying the first medical image at a local workstation; monitoring a predetermined status indication at the local workstation, the predetermined status indication being associated with the first medical image; displaying at least one first model output corresponding to the first medical image from the first remote computer upon the local workstation detecting a change in the predetermined status indication; capturing at least one first interaction with the at least one first model output at the local workstation; and transmitting the at least one first interaction with an identification pointing to the first medical image to the first remote computer.

In at least some embodiments, the techniques described herein relate to a system, including: at least one processor; and a memory in communication with the at least one processor and storing instructions that, when executed by the at least one processor, cause the at least one processor to: transmit a first medical image of a first subject case to a first remote computer with a first machine-learning (ML) model for processing the first medical image; display the first medical image at a local workstation; monitor a predetermined status indication at the local workstation, the predetermined status indication being associated with the first medical image; display at least one first model output corresponding to the first medical image from the first remote computer upon the local workstation detecting a change in the predetermined status indication; capture at least one first interaction with the at least one first model output at the local workstation; and transmit the at least one first interaction with an identification pointing to the first medical image to the first remote computer.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 is a block diagram illustrating an exemplary AI-enabled radiology system according to one or more embodiments of the present disclosure.

FIG. 2 is a flowchart illustrating an AI-enhanced radiology workflow according to one or more embodiments of the present disclosure.

FIG. 3 is a block diagram illustrating an exemplary user interface (UI) of a radiologist report template according to one or more embodiments of the present disclosure.

FIG. 4 is a block diagram illustrating components of an AI-enhanced radiology system according to one or more embodiments of the present disclosure.

FIG. 5 is a flowchart illustrating an AI-enhanced medical imaging interpretation workflow according to one or more embodiments of the present disclosure.

FIG. 6 is a first summary diagram of the features of the MFILE-DCS software in accordance with one of more embodiments of the present disclosure.

FIG. 7 is a second summary diagram of the features of the MFILE-DCS software in accordance with one of more embodiments of the present disclosure.

FIG. 8 is a screenshot of an exemplary subject medical record with errors detected by the MFILE-DCS software in accordance with one or more embodiments of the present disclosure.

FIG. 9 is a third summary diagram of the MFILE-DCS software in accordance with one or more embodiments of the present disclosure.

FIG. 10 is a flowchart illustrating an AI-enhanced radiologist's interpretation process in accordance with one or more embodiments of the present disclosure.

FIG. 11 is a block diagram illustrating an exemplary computer-based system/platform in accordance with one or more embodiments of the present disclosure.

### DETAILED DESCRIPTION

A technical problem faced in the field of medical imaging interpretation in the age of growing popularity of machine-learning (ML) is how to integrate AI-based medical diagnosis system with existing medical imaging interpretation system. In at least some embodiments, the present disclosure details at least one hardware and software platform that integrates and evaluates diverse AI tools within medical imaging interpretation workflows at a time after a report is generated but prior to the report is signed off. An example that involves such medical imaging interpretation is radiology which is used as an exemplary application according to one or more principles of the present disclosure described hereinbelow.

In at least some embodiments, the hardware and software platform is designed to integrate with existing radiology workflows and presents AI generated findings to radiologists after their independent interpretation and before final report sign-off, allowing for unbiased verification of AI results without introducing cognitive anchoring or workflow disruption. This hardware and software platform reduces costs associated with disparate integrations, minimizes disruptions, and measures value of the AI tools.

In at least some embodiments, the hardware and software platform acts as an intermediary layer within the Picture Archiving and Communication System (PACS) or Radiology Information System (RIS) environment, automatically retrieving AI results from one or more vendors in the background. In at least some embodiments, these results are held until the radiologist completes their preliminary interpretation, at which point the software platform surfaces the model outputs and/or insights in a structured, interactive format. In at least some embodiments, this timing ensures that radiologist autonomy is preserved, while still enabling efficient cross-verification to improve diagnostic accuracy and confidence.

Furthermore, in at least some embodiments, the hardware and software platform establishes a continuous feedback loop by aggregating and anonymizing data on diagnostic agreement, discrepancies, and user interaction. In at least some embodiments, this performance data is securely shared with participating artificial intelligence (AI) vendor(s), enabling targeted refinement and retraining of algorithms based on real-world clinical use. As a result, in at least some embodiments, the hardware and software platform not only supports safe clinical adoption of AI but also drives a data-informed evolution of AI tools toward greater precision and clinical utility.

In at least some embodiments, the present disclosure details a just-in-time system that maximizes machine-learning (ML) value and measures the true value of AI without subject risk. The just-in-time system can swap AI vendors as well as create valuable data asset for the AI vendors.

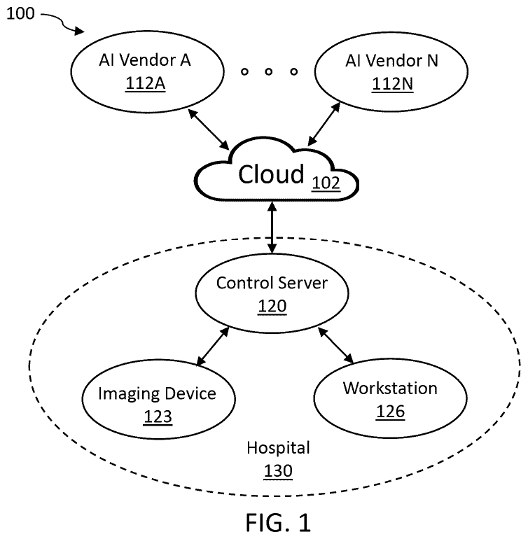

FIG. 1 is a block diagram illustrating an exemplary AI-enabled radiology system 100 according to one or more embodiments of the present disclosure. In at least some embodiments, the AI-enabled radiology system 100 includes an exemplary imaging device 123, a control server 120 and a workstation 126 in an exemplary hospital 130. The imaging device 123 is a diagnostic tool that can take images of a subject's body. The imaging device 123 can be an X-ray machine, an ultrasound machine, a computed tomography (CT) scanner, a magnetic resonance imaging (MRI) scanner, a position emission tomography (PET) scanner and an echocardiography machine. The X-rays are used to produce images of structures within the body, particularly useful for viewing bones and detecting fractures. The ultrasound machine uses high-frequency sound waves to create images of organs and structures inside the body, often used in obstetrics and cardiology. The CT scanner combines X-ray images taken from different angles to create cross-sectional images of the body, providing more detailed information than regular X-rays. The MRI scanner uses strong magnetic fields and radio waves to generate detailed images of organs and tissues, particularly useful for brain, spine, and joint imaging. The PET scanner uses a small amount of radioactive material to highlight areas of high metabolic activity, often used in cancer diagnosis and monitoring. The echocardiography machine uses ultrasound to create images of the heart, helping to assess its function and structure.

Referring again to FIG. 1, in at least some embodiments, the exemplary control server 120 is configured to execute the aforementioned software platform to provide the enhanced radiology workflow. In at least some embodiments, the control serve 120 is connected with both the imaging device 123 and the workstation 126. The control server 120 may perform the functions of image acquisition and monitoring, quality control, communication hub, and safety management. In the image acquisition and monitoring, technicians operate the imaging device 123 from the control server, ensuring proper image capture and subject safety. In the quality control, the control server 120 can monitor the quality of images in real-time, adjusting as needed to ensure diagnostic accuracy. As a communication hub, the control server may act as a central point for communication between the imaging device 123, the workstation 126 and outside world, e.g., AI vendors A-N 112A-112N, via the cloud 102. In at least some embodiments, the number of vendors is less than 5. In at least some other embodiments, the number of vendors is in a range of 5 to 10.

Referring again to FIG. 1, In at least some embodiments, the exemplary workstation 126 is a specialized setup designed for viewing medical images taken by the imaging device 123 and creating diagnostic reports by a radiologist. In at least some embodiments, the workstation 126 includes multiple high-resolution monitors to view detailed medical images and a Picture Archiving and Communication system (PACS) which is a software application that allows radiologists to access, view, and manipulate medical images. In at least some embodiments, the PACS is enhanced by integrating the aforementioned software platform. In at least some embodiment, the PACS may be integrated with a large language model, so that the PACS can receive instructions in voice, gesture, or both, and recognize content of a report as it is being drafted.

In at least some embodiments, the PACS is also integrated with the control server 120 to streamline workflow and improve efficiency. In at least some embodiments, the PACS allows a radiologist to select a particular AI vendor 112i for image recognition and diagnostic assistance. In at least some embodiments, the PACS displays an AI generated diagnostic report in the workstation 126 in response to detecting a local report generated at the workstation 126. In at least some embodiments, multiple AI vendors A-N 112A-112N can be employed to separately generate AI analysis on particular medical image(s). The PACS unifies outputs from disparate AI vendors into a single, cohesive interface, normalizing, and presenting results in a structured format. The PACS also can also compare the outputs from disparate AI vendors. This orchestration of multiple vendor results by the PACS occurs contextually (e.g., activated only when multiple AI results are presented) and on-demand (e.g., the radiologist has control over the activation of such orchestration). In at least some embodiments, the PACS allows the radiologist to switch AI vendors.

In at least some embodiments, the aforementioned software platform that is integrated in the PACS is built on a modular and interoperable framework, that can seamlessly integrate with both pixel-based AI systems (e.g., medical image processing) and non-pixel-based AI systems (e.g., large language models), and is compatible with leading platforms such as Epic, Nuance PowerScribe, AIDOC, and RAPID. This flexibility enables rapid deployment across diverse enterprise environments.

In at least some embodiments, the PACS collects feedback regarding the AI generated diagnostic report from the workstations and provide the feedback to the corresponding AI vendor 112i, wherein i is an integer between A and N, for further training the ML model. In at least some embodiments, before transmitting the feedback to the AI vendor 112i, the data may be anonymized on diagnostic agreement, discrepancies, and/or user interaction and identification. Only performance data is aggregated and shared with the AI vendor 112i.

Referring again to FIG. 1, in at least some embodiments, the AI vendors A-N 112A-112N are connected to the control server 120 via the cloud 102. Each of the AI vendors A-N 112A-112N may offer unique solutions to enhance diagnostic accuracy and efficiency. The control server 120 may select a different AI vendor i 112i for diagnosing a different medical image. In at least some embodiments, the con

In at least some embodiments, each AI vendor i 112i may develop and train its ML model(s) by collecting vast amounts of medical imaging data. Using these data, the AI vendor i 112i trains algorithms to recognize patterns and anomalies in medical images. The algorithm training involves supervised learning, where the algorithm learns from labeled data, and unsupervised learning, where it identifies patterns without labeled data.

In at least some embodiments, the ML model(s) may be pixel-based in which processing and analyzing images are at pixel level to perform various tasks. In the pixel-based system, images are captured using cameras or other imaging devices. Each image is made up of pixels, which are the smallest units of the image. In at least some embodiments, the AI system preprocesses the image to enhance quality, remove noise, and adjust brightness and contrast. In at least some embodiments, this preprocessing step ensures that the image is in a sufficient condition for analysis that analyzes the pixels to extract features such as, without a limitation, edges, textures, and/or patterns. The preprocessing involves techniques like edge detection, color analysis, and texture mapping. In at least some embodiments, using machine learning algorithms, the exemplary AI system is configured to identify patterns and objects within the image. In at least some embodiments, the exemplary AI system is configured to, at least, detect anomalies, and/or identify specific object(s). The AI system classifies the identified patterns and provides insights based on the analysis. For example, in medical imaging, it may detect tumors or other abnormalities.

In at least some embodiments, the AI vendors A-N 112A-112N are integrated with software applications in the control server 120 as well as the PACS in the workstation 126, so that the AI tools provided by the AI vendors A-N 112A-112N can be accessible within the radiologist's workflow.

In at least some embodiments, the AI algorithms provided by the AI vendors A-N 112A-112N can analyze medical images in real-time, providing immediate feedback to radiologists with hospital 130. In at least some embodiments, the provisioning of multiple AI algorithms facilitating in identifying critical conditions such as, without limitation, tumors, fractures, hemorrhages, etc.

In at least some embodiments, the AI tools developed by the AI vendors A-N 112A-112N can detect and/or highlight abnormality(ies) in medical images, such as, without limitation, lung nodules, brain tumors, cardiac issues, etc. In at least some embodiments, the identification of abnormality(ies) can assist radiologists in making more accurate diagnoses.

In at least some embodiments, the AI tools developed by the AI vendors A-N 112A-112N can assist in generating preliminary reports, which radiologists can then review and finalize. The preliminary reports can speed up the reporting process and reduce the workload on radiologists.

In at least some embodiments, the AI system developed by the AI vendors A-N 112A-112N may continuously learn from new data and feedback from radiologists, improving their accuracy and reliability over time.

In at least some embodiments, the workstation 126 employs a status indicator, for example, LED lights that can provide visual feedback indicating the PACS software status, such as, without limitation, “Scanning”, “Errors Found”, “All Clear,” or any other similar statuses.

In at least some embodiments, the workstation 126 is connected to the control server via an Ethernet and/or Wi-Fi connection.

In at least some embodiments, the PACS software is programmed to provide a feedback mechanism. After the AI vendor 112i can send a signal to the workstation to inform the radiologist that the model outputs are available.

In at least some embodiments, the workstation 126 is programmed to provide customizable hotkeys for frequently used functions, like save draft, retrieve model outputs, sign out, or lock the workstation 126.

In at least some embodiments, the control server 120 is connected to multiple workstations, and each workstation is portable to allow radiologists to carry the workstation to different reading rooms.

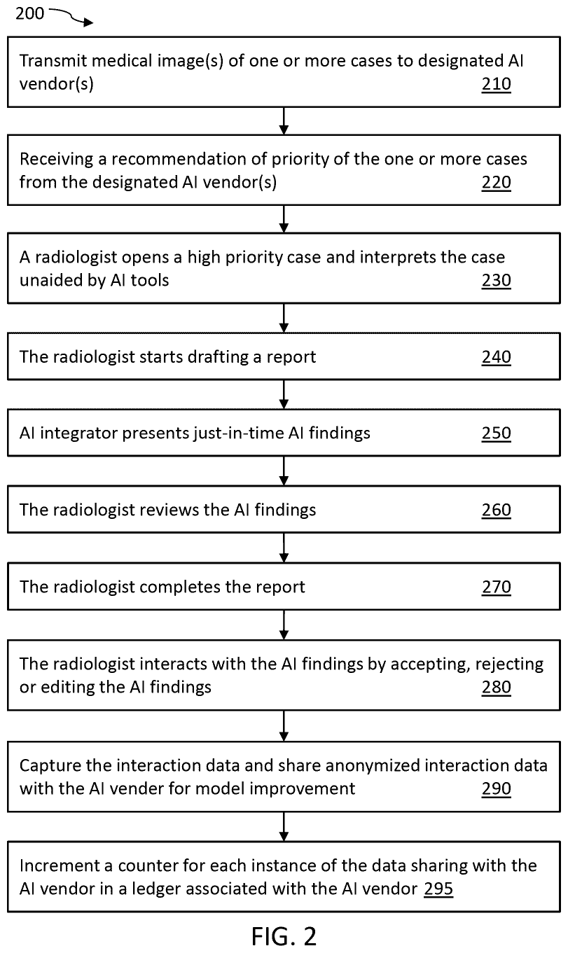

FIG. 2 is a flowchart illustrating an AI-enhanced radiology workflow 200 according one or more embodiments of the present disclosure. The workflow 200 includes exemplary steps 210-295 as described herein below.

In step 210, in at least some embodiments, the workflow 200 involves transmitting, by the control server 120, medical image(s) of one or more cases to designated AI vendor(s) 112i.

In step 220, in at least some embodiments, the workflow 200 involves receiving, by the control server 120, a recommendation of priority of the one or more cases from the designated AI vendor(s). In at least some embodiments, the designated AI vendor(s) generate the recommendation by comparing seriousness the cases based on AI-performed diagnoses. A more serious case receives higher priority, so that the radiologist may first take on the more serious case.

In step 230, in at least some embodiments, the radiologist opens a high priority case recommended by the AI vendor(s). The corresponding medical images are then loaded to the workstation 126 for the radiologist to study and interpret unaided by AI tools.

In step 240, after studying and interpreting the one or more medical images, the radiologist may start drafting a report on the workstation 126. In at least some embodiments, the workflow 200 may deploy a large language model to assist the report drafting and monitor the progress thereof.

In step 250, after the report drafting having progressed to a predetermined stage, an AI integrator deployed in the workflow 200 may present model outputs in the one or more medical images from the designated AI vendor. In an embodiments, the model outputs may be presented to the workstation 126 just in time before the radiologist is ready to sign the report. In another embodiments, the model outputs may be presented to the workstation 126 just in time when the radiologist has finished interpreting the one or more medical images and is at the start of the report drafting. The model outputs may contain information generated by the AI to assist the report drafting. In at least some embodiments, the workstation presents a button on a user interface (UI) of the workstation 126. By pressing the button, the radiologist can retrieve the model outputs at any time. However, in at least some embodiments, the workflow 200 is programmed to have a rule that the model outputs can only be retrieved after a diagnostic conclusion is entered in the report. In at least some embodiments, the workflow 200 provides a report template with text boxes for various items. When the text box for the diagnostic conclusion is filled, for example, with a predetermined number of characters, the model outputs will be automatically retrieved and displayed on the workstation 126.

In step 260, the radiologist may review the model outputs on the workstation 126.

In step 270, the radiologist may complete the report after reviewing the model outputs.

In step 280, the radiologist may interact with the model outputs by accepting, rejecting, or editing the model outputs.

In step 290, the workflow 200 may capture the radiologist's interaction data and share the anonymized interaction data with the AI vender for model improvement.

In step 295, in at least some embodiments, the workflow 200 increments a counter by, for example, one for each instance of the data sharing with the AI vendor in a ledger associated with the AI vendor 112i, so that the hospital 130 may be compensated by the AI vendor 112i for providing the feedback data. In at least some embodiments, the ledger is stored with the control server 120.

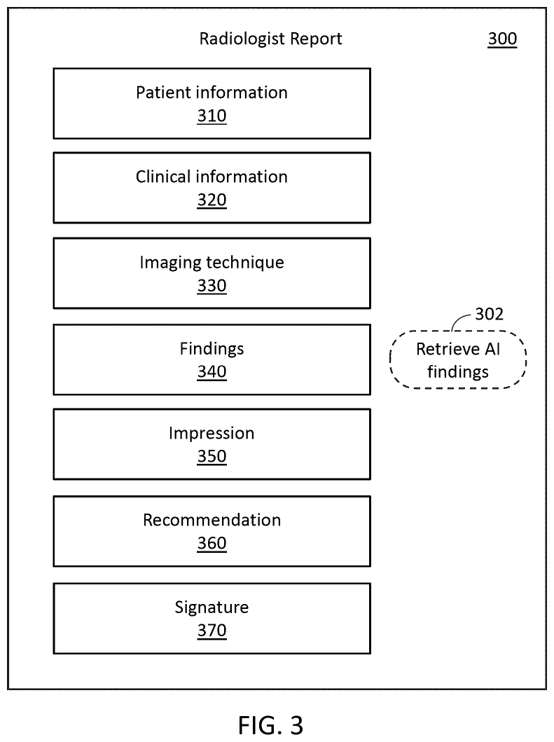

FIG. 3 is a block diagram illustrating an exemplary user interface (UI) 300 of a radiologist report template according to one or more embodiments of the present disclosure. The exemplary UI 300 includes a text box 310 for entering subject information, a text box 320 for entering clinical information, a text box 330 for entering imaging technique, a text box 340 for entering radiologist finds, a text box 350 for entering radiologist impression, a text box 360 for entering radiologist recommendation, a signature box 370, and a button 302 for entering command to retrieve model output from a designated AI vendor. In at least some embodiments, the button 302 is invisible when the UI 300 is first loaded until a predetermined text box is filled to a predetermined extent. For example, the findings text box 340 is programed to detect a number of characters being entered therein. If the number reaches a predetermined threshold, for instance, 100 characters, the button 302 will become visible and active. In another example, the button 302 is programmed to float with a text cursor on the UI 300. For example, when the text cursor is in the impression text box 350, the button 302 will appear next to the impression text box 350.

In at least some embodiments, the UI 300 is programmed to detect an activation of the signature box 370. When the signature box 370 is activated and the model outputs have been retrieved before in the same UI session, the button 302 becomes invisible and inactive again.

In at least some embodiments, the UI button 302 is replaced with a physical switch (not shown) that is connected to the workstation 126 via, for example, a USB port. Once the switch is triggered, the workstation 126 will generate a request to retrieve the model outputs from the designated AI vendor.

Although the present disclosure details exemplary radiology workflows, the architectures and processes of the present disclosure is adaptable to other diagnostic fields such as pathology, dermatology, ad ophthalmology—where visual pattern recognition and AI decision support are employed.

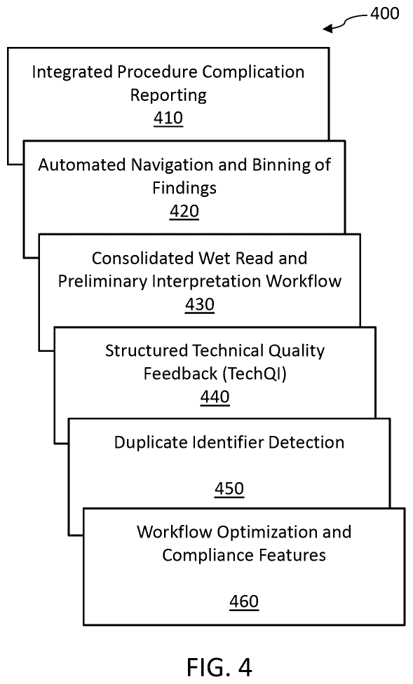

FIG. 4 is a block diagram illustrating components of an AI-enhanced radiology system 400 according to one or more embodiments of the present disclosure. The system 400 is designed to improve radiology operations, quality, and compliance within an AI-enabled radiology environment. The figure enumerates several functional groupings or components 410-460, each represents a specific category of workflow intelligence or automation provided by the system 400.

In at least some embodiments, the component 410 represents integrated procedure complication reporting. The component 410 enables systematic capture and reporting of procedural complications, helping ensure completeness, consistency, and downstream quality analysis. In implementations, the component 410 provides a graphical user interface (GUI) element that allows procedural complications to be documented in real time, either during the initial interpretation or through a subsequent addendum. The interface is integrated directly into the dictation workflow and includes a dedicated complication reporting control, such as a button or selectable field, that prompts the user to record relevant details. When activated, the interface presents required input fields for specifying the complication type, its category, and accompanying descriptive comments. Built-in validation logic ensures that all required information is completed before the entry can be submitted, preventing incomplete or inconsistent reports. By enabling structured complication reporting within the existing workflow and without reliance on external systems, component 410 enhances documentation consistency, improves compliance, and supports downstream auditing and quality review.

In at least some embodiments, the component 420 represents automated navigation and binning of findings. The component 420 automatically organizes and categorizes radiology findings, allowing radiologists to navigate large or complex cases more efficiently. In implementations, the component 420 incorporates automated binning algorithms designed to identify and organize actionable and incidental findings within clinical reports. When relevant findings are detected, the system automatically classifies them into predefined categories, such as critical, urgent, or incidental, based on established criteria. In parallel, associated navigation panels are dynamically updated to reflect these classifications, enabling users to efficiently access and manage necessary follow-up actions. By automating the categorization and surfacing of findings within the workflow, the component 420 minimizes the need for manual tracking and supports more efficient longitudinal subject care.

In at least some embodiments, the component 430 represents consolidated wet read and preliminary interpretation workflow. The component 430 supports early or “wet read” interpretations and integrates them into a consolidated preliminary reporting workflow, reducing fragmentation between initial and finalized reads. In implementations, the component 430 delivers a unified interface for managing both wet read discrepancies and resident preliminary interpretations within a single, cohesive workflow. The interface enables real-time comparison between preliminary interpretations and the finalized report, allowing users to quickly identify areas of alignment or discrepancy. Interactive controls are provided to explicitly indicate agreement or disagreement with preliminary findings, streamlining review and documentation. As discrepancies are resolved, the system can automatically trigger notifications to the appropriate clinical teams, ensuring timely awareness of critical updates. By consolidating these capabilities into a single interface, this embodiment removes redundant steps, improves clarity during interpretation review, and promotes efficient, timely communication across care teams.

In at least some embodiments, the component 440 represents structured technical quality feedback (TechQI). The component 440 provides structured feedback on technical image quality, supporting continuous process improvement for technologists and imaging systems. In implementations, the component 440 enables radiologists to provide structured feedback on image acquisition quality across a wide range of imaging modalities, including CT, MR, ultrasound, mammography, nuclear medicine, PET, computed radiography, and dual-energy X-ray absorptiometry (DEXA). The interface supports a predefined taxonomy of quality categories, such as suboptimal image quality, protocol deviations, or positive performance, allowing feedback to be captured consistently and efficiently. The system further supports closed-loop communication by automatically transmitting this feedback to technologists and modality leads through notification mechanisms. By facilitating timely, structured exchange between radiologists and imaging staff, this embodiment promotes higher imaging standards and supports ongoing quality improvement initiatives.

In at least some embodiments, the component 450 represents duplicate identifier detection. The component 450 detects potential duplicate subject or study identifiers, helping prevent reporting or attribution errors that could affect subject safety and data integrity. In implementations, the component 450 implements logic to detect the presence of duplicate subject identifiers, such as multiple medical record numbers (MRNs) or enterprise subject identifiers (EPIs) associated with the same individual. When such duplicates are identified, the system proactively surfaces prior imaging studies linked to the alternate identifiers, providing the interpreting physician with a more complete clinical history. At the same time, alerts are generated to notify the user of potential data fragmentation across records. By bringing these discrepancies to attention during interpretation, this embodiment helps reduce diagnostic delays, supports more informed clinical decision-making, and mitigates subject safety risks associated with incomplete or fragmented data.

In at least some embodiments, the component 460 represents workflow optimization and compliance features. The component 460 represents broader tools that monitor, optimize, and enforce the best workflow practices and regulatory or institutional compliance requirements. In implementations, the component 460 expands the capabilities of the system 400 by supporting standardized reporting and operational integration. For example, the component 460 enforces the use of standardized reporting templates for both oncology and non-oncology cases, promoting consistency and completeness in clinical documentation. The system may also integrate with external electronic health record (EHR) systems to support provider mapping and alignment of billing codes, thereby streamlining downstream administrative and revenue cycle workflows. In addition, real-time monitoring of productivity metrics, such as relative value units (RVUs), is provided to give clinicians and administrators visibility into performance and workload. Together, these features help improve reporting quality, strengthen interoperability, and support operational efficiency across the organization.

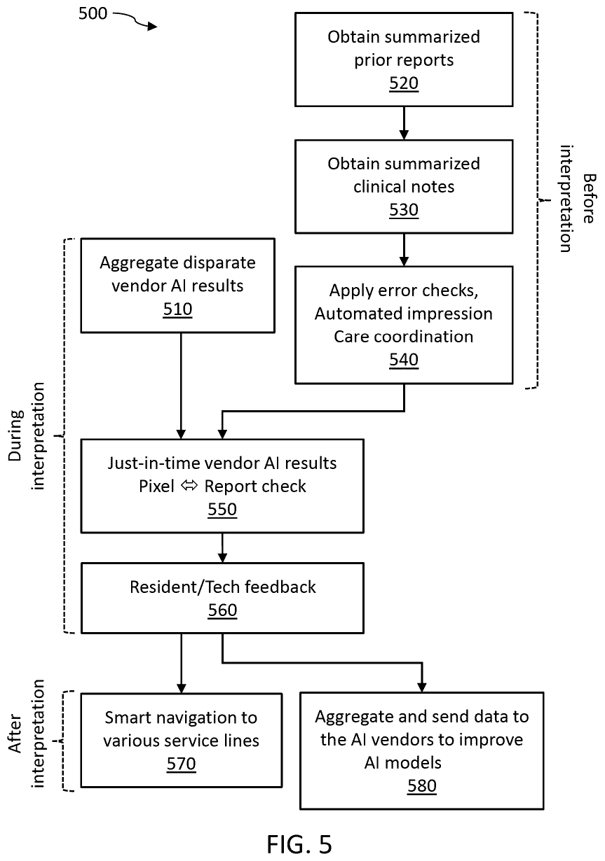

FIG. 5 is a flowchart illustrating an AI-enhanced medical imaging interpretation workflow 500 according to one or more embodiments of the present disclosure. The workflow 500 includes exemplary blocks 510-580 as described below. In embodiments, the blocks 520-540 belong to a before-interpretation step; the blocks 510, 550 and 560 belong to a during-interpretation step; and the block 570 and 580 belong to an after-interpretation step.

In block 510, the workflow 500 includes aggregating disparate vendor AI results for imaging of a particular subject. The AI results are provided to the radiologist just-in-time in block 550.

In block 520, the workflow 500 includes obtaining summarized prior reports. In an embodiment, the prior reports pertain to the particular subject. In another embodiment, the prior reports pertain to a particular illness of multiple subjects.

In block 530, the workflow 500 includes obtaining summarized clinical notes. In an embodiment, the clinical notes pertain to the particular subject. In another embodiment, the clinical notes pertain to a particular illness of multiple subjects. In at least some embodiments, both the prior reports and the clinical notes assist the radiologist to make a diagnosis.

In block 540, the workflow 500 includes applying error checks on the automated impression and care coordination. In at least some embodiments, the automated impression and care coordination refer to the fact that the radiologist uses an AI assistant to automatically generate the impression section and care coordination section of a radiology report based on the radiologist's dictated findings. In at least some embodiments, there are 10-20 checking items in the error checks. In at least some embodiments, there are 20-40 checking items in the error checks. In at least some embodiments, there are 40-60 checking items in the error checks.

In block 550, both the AI results from block 510 are provided to the radiologist just in time after the creation of the automated impression and coordination, so that the radiologist can compare the automated impression and coordination with the AI results. In at least some embodiments, the digital image pixels are compared with the report from the AI results to help identify any discrepancies or artifacts that may affect the diagnostic quality of the image.

In block 560, a resident or a technical personnel provides feedback to the AI results based on, for example, the radiologist's diagnosis.

In block 570, the workflow 500 includes a smart navigation of the subject to various service lines for treatment.

In block 580, the workflow 500 includes aggregating and sending the feedback data to the AI vendors to improve their ML models.

In at least some embodiments, the control server 120 may execute a middleware platform between a radiologist's primary reporting system and the final report-signing interface. The middleware platform may capture radiology cases after interpretation but before final signing, may run non-AI and AI analysis, may display a reconciliation view to the radiologist, and may collect structured feedback to continuously improve AI performance.

In at least some embodiments, the middleware platform may require general-purpose computing hardware capable of running containerized services, such as Docker or equivalent. The hardware may include a multi-core CPU, at least 8 GB RAM, IDE, or SSD storage, viewing monitors, keyboard, and mouse. The middleware platform may be deployed on a Windows-based workstation and may be installed on a local appliance, a connected data center, a cloud-hosted virtual machine, or a hybrid environment. The middleware platform may require network connectivity to a structured report generator, a workflow or worklist orchestration tool, a HIPAA compliant large language model API, installed and in-scope pixel and non-pixel based AI tools provided by external and internal vendors, a resource scheduling system API, robotic process automation software, a database such as Oracle or MSSQL, exchange and messaging clients, customized fee structures and examination sets, and teleradiology service connectivity.

In at least some embodiments, the middleware platform may include an automation layer that may manage data extraction and movement across systems. This automation layer may be implemented using robotic process automation tools or custom scripts in programming languages such as Python, Java, or PHP.

In at least some embodiments, the middleware platform may include an AI integration layer that may handle calls to one or more inference engines for internal or external computer vision analysis. The AI integration layer may be compatible with on-premise ML models, containerized AI endpoints, or cloud-based AI services using RESTful APIs.

In at least some embodiments, the middleware platform may include a case broker module that may manage case eligibility for the hardware and software platform. The case broker module may review and adjust choices and options based on case modulation, such as only unsigned reports from eligible modalities.

In at least some embodiments, the middleware platform may include a presentation layer that may display a structured comparison between radiologist findings and non-AI and AI output. The presentation layer may allow for inline feedback, such as agree or disagree selections, annotation on images, free-text clarification, and activation of specific modules.

In at least some embodiments, the middleware platform may include a feedback engine that may store structured and unstructured user feedback. The feedback engine may send deidentified data and annotated identified data back to AI vendors or local models for performance tuning.

In at least some embodiments, the middleware platform may be set up by connecting to source systems, such as configuring automated data retrieval from PACS, connecting to the reporting system's output feed, and connecting to robotic process automation software. The middleware platform may define rules to identify which studies may receive which modules from the hardware and software platform, based on modality, report status, time rules, user-based rules, subject class rules, non-AI and AI rules, priority status, external system exam statuses, and navigation binning.

In at least some embodiments, the middleware platform may forward study reports and feedback to the AI vendor, either in batch or per-case. The middleware platform may capture AI output, including navigation binning and navigation.

In at least some embodiments, the middleware platform may display an intermediate review view that may show side-by-side the radiologist report text and model outputs, with visual cues and structured labels. The radiologist may approve, amend, or flag the non-AI and AI results, and the middleware platform may capture user responses for each suggested finding.

In at least some embodiments, the middleware platform may allow the final report to be unchanged or edited by the radiologist based on non-AI and AI insights. The middleware platform may log feedback, link it to the study ID, or anonymize it, and store it for auditing and reviewing purposes. The feedback may be routed for vendor quality assurance or training pipelines.

In at least some embodiments, the middleware platform may remove or encrypt protected health information before AI vendor communication, in accordance with legal agreements. The middleware platform may support role-based access controls, logging of all feedback and suggested edits, and options to anonymize or pseudonymize cases for research or AI tuning.

In at least some embodiments, the middleware platform may support multiple ML models per modality, may operate asynchronously or synchronously, and may be deployed fully on premises, as a hybrid cloud tool, or as a plugin into existing reporting platforms.

There have been recorded instances where errors in medical files have been attributed to unnecessary severe injuries and deaths of subjects. One specific type of medical error or serious adverse event called a “sentinel event” may be defined as “an unexpected occurrence involving death or serious physiological or psychological injury, or the risk thereof.” Such events are called “sentinel” because they signal the need for immediate investigation and response.

For example, medical errors (such as wrong-site surgery) that should never occur may be attributed, for example, to an error in laterality in X-ray images, where a particular anatomical feature and/or a pathological feature such as a tumor, is marked as being on the wrong side of the subject's body. For example, the feature of interest may really be on the left side of the subject but marked as being on the right side in the medical report and/or on the medical image that may cause a surgeon, for example, to operate on the wrong side of the subject due to the laterality error. Furthermore, if these medical file errors are not discovered before the medical file is finalized and signed, these medical file errors may become part of the subject's file that typically cannot be changed and/or deleted if later discovered. Then these errors may propagate into future medical reports if not corrected.

Note that the terms medical file, medical record, medical report, and report may be used interchangeably herein.

Embodiments of the present invention herein disclose computer-based methods and systems for automated medical file error detection and correction. An automated medical file error detection and correction system (MFILE-DCS) may be implemented as a computer program executed by the computer associated with a healthcare provider such as physician. The MFILE-DCS software may be executed in a computer enterprise, on a computer server, and/or on a local machine associated with the healthcare provider.

In at least some embodiments, the MFILE-DCS software may invoke a systematic and comprehensive check for a growing number of quality, safety, financial and efficiency measures across various underlying systems. It may focus on preventing errors before a radiology report or other types of medical reports may be signed and become legal documents which may not be changed (only addended). This check may be prospectively performed with the intention to highlight and resolve errors before they become a permanent part of the subject's records.

In at least some embodiments, the MFILE-DCS software may be unique and novel in that in addition to looking at textual material contained in the report and report headers, the MFILE-DCS software may integrate information from external systems such as AI algorithm outputs, radiology information systems (RIS), electronic health records (EHR), and credentialing/privileges databases. The MFILE-DCS software may consider multi system and multi factorial events to highlight and modify those items before a report is signed.

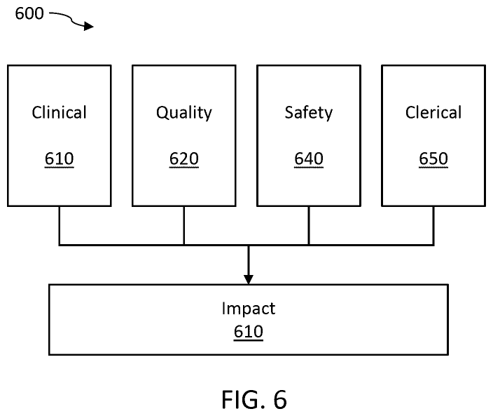

FIG. 6 is a first summary diagram 600 of the features of the MFILE-DCS software in accordance with one of more embodiments of the present disclosure. The MFILE-DCS software may be configured to detect errors in subject medical records in a clinical 610 class of medical record errors, a quality 620 class of medical record errors, a safety 630 class of medical record errors, and a clerical 640 class of medical record errors. These errors can cause impact 650.

As an example, detecting clinical 610 class includes: checking for any STAT out-subject exam and telling radiologist to call the technology team since the subject might still be waiting; checking for ED Wet Reads (Powerscribe (any dictation system) custom field); and checking AI tools including AIDOC.

As an example, detecting quality 620 class includes: checking if subject is a Medicare (or other insurance provider) enrollee to verify signer has rights to sign report; checking for any Medicalis (any worklist tool or RIS worklist tool) prelim notes, displaying them; checking for accessions linked in Medicalis (any worklist tool or RIS worklist tool), if one accession in report is linked to another, gives warning that other accessions should also be included in report; checking if Medicalis (any worklist tool or RIS worklist tool) sub-specialties for multi accession reports are ok (example a NEURO shouldn't go with a MAMMO, will trigger warning); and checking for CT exams with contrast and subject less than 3 years of age, if so certain text should be in report.

As an example, detecting safety 630 class includes: checking for left/right in report, checking if exam is specifically for a ‘left’ or ‘right’ body part; gender checking; looking for words ‘Male’ and ‘Female’ in report to make sure matches gender of subject; looking for keywords (body parts, organs) in report to make sure they are for the right gender.

As an example, detecting clerical 140 class includes: checking for un-resolved problems in Medicalis (any worklist tool or RIS worklist tool) for any accessions; checking if accession is still in scheduled status; checking to make sure Safe Sign was used when report gets signed; checking start dates/privilege/credential dates to verify provider can provide care at the site of the report; moonlighting tracking; resident agree/disagree tracking and feedback.

As an example, the impact 650 includes: similar tools are retroactively reviewing signed reports and highlighting errors, but very few are preventing them. MFILE-DCS is unique and novel in that in addition to looking at textual material contained in the report and report headers, MFILE-DCS integrates information from external systems such as AI algorithm outputs, radiology information systems (RIS), electronic health records (EHR), and credentialing/privileges databases. MFILE-DCS for Reports looks at multi-system and multi-factorial events to highlight and modify those items before a report is signed.

In 2008, per the National Library of Medicine, medical errors cost the United States $19.5 billion. About 87 percent or $17 billion were directly associated with additional medical cost, including: ancillary services, prescription drug services, and in-subject and out-subject care.

In at least some embodiments, the MFILE-DCS invokes a systematic and comprehensive check for a growing numbers of quality, safety, financial and efficiency measures across various underlying systems. It focuses on preventing errors before a report or other types of documents are signed and become legal documents which cannot be changed (only addended). This check is prospectively performed with the intention to highlight and resolve errors before they become a permanent part of the subject's records.

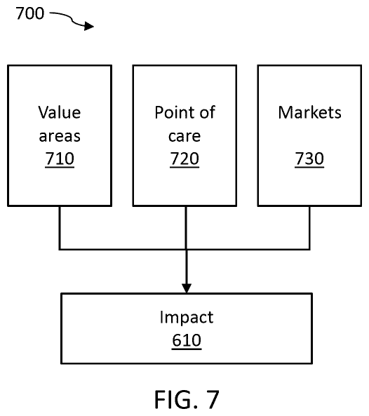

FIG. 7 is a second summary diagram 700 of the features of the MFILE-DCS software in accordance with one of more embodiments of the present disclosure. The features of the MFILE-DCS software includes value areas 710, point of care (P.O.C) 720 and markets 730. These features make impact 650 as described above.

As an example, the value areas 710 include clinical, financial, quality, safety, and clerical education areas.

As an example, the P.O.C. include more than 10 million reports and about 4.9 million checks-caught-before-signing.

As an example, the markets include radiology, labs, point of care ultrasound and electronic medical records (EMRs).

In at least some embodiments, the MFILE-DCS invokes a systematic and comprehensive check for a growing numbers of quality, safety, financial and efficiency measures across various underlying systems. It focuses on preventing errors before a report or other types of documents are signed and become legal documents which cannot be changed (only addended). The system overlays the vendor's “sign or complete” button with the Safe Sign button. This check is prospectively performed with the intention to highlight and resolve errors before they become a permanent part of the subject's records.

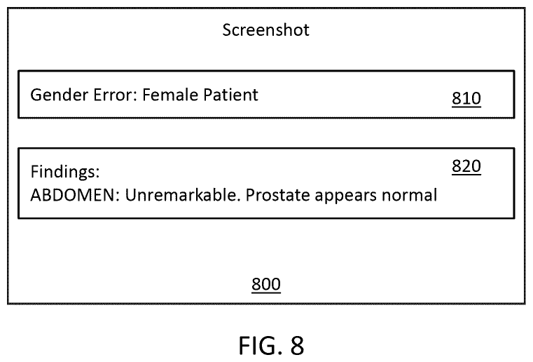

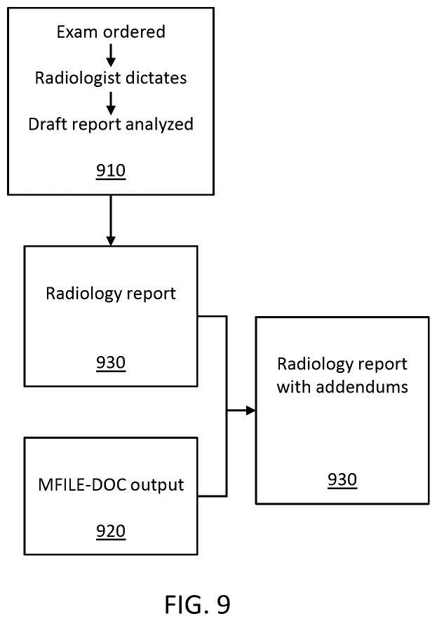

In at least some embodiments, the medical record error detection algorithms used by the MFILE-DCS software may include but are not limited to rules-based medical record error detection, algorithm-based medical record error detection, and/or machine learning model (MLM) AI-based medical record error detection. In the case of the MLMs, the MLM may be trained on training datasets including input data features from each class type of medical record error from the clinical 610 class, the quality 620 class, the safety 630 class, and/or the clerical 640 class of medical record errors. The output data features of the training datasets may include a location of the medical record errors in the medical record (e.g., by highlighting keywords or providing any suitable location indicia for display—see FIG. 8) for the user (e.g., healthcare provider) to fix, and/or an automated correction of the medical record errors. This may occur before the medical records are digitally signed (e.g., Safe Sign) and finalized or may add medical report addendums if the report was finalized. In other embodiments, the MLM may be further configured to identify other medical problems and/or pathologies not detected by the healthcare provider and not register in the medical report. (See as a non-limiting example, FIG. 9—AI detected Liver Iron Overload)

In at least some embodiments, the MFILE-DCS software may allow a user to correct the detected medical record errors as shown on a user display (referring to FIG. 8). In other embodiments, the MFILE-DCS software may be configured to implement an automated correction of the detected medical record errors.

In at least some embodiments, the MFILE-DCS software may be configured to check for left and right in the medical report and then cross-check if the exam was intended for a left or a right body part.

In at least some embodiments, the MFILE-DCS software may be configured to check gender by looking for “male” and “female” in the medical report and cross-checks if it matches the gender of the subject. In other embodiments, the MFILE-DCS software may be configured to check gender by looking for keywords in the medical report (e.g., gender-specific body parts, organs) and to cross-check that the gender-specific body parts are consistent with the subject's gender.

In at least some embodiments, the MFILE-DCS software may be configured to examine emergency department (ED) wet reads of X-rays images, for example, custom fields within Powerscribe. In other embodiments, the MFILE-DCS software may be configured to examine wet reads in Medicalis (e.g., for NWCH and PHELPS).

In at least some embodiments, the MFILE-DCS software may be configured to examine any Medicalis preliminary notes such as for example, any worklist tool and/or RIS worklist tool, and to display them.

In at least some embodiments, the MFILE-DCS software may be configured to check for accessions linked to Medicalis, such as for example, any worklist tool and/or RIS worklist tool. If one accession in the subject's medical report is linked to another, the MFILE-DCS software may generate a warning that other accessions should also be included in report.

In at least some embodiments, the MFILE-DCS software may be configured to check if Medicalis, such as for example, any worklist tool and/or RIS worklist tool, sub-specialties for multi accession reports are consistent. For example, a NEURO is inconsistent with a MAMMO, such that the MFILE-DCS software may trigger a warning.

In at least some embodiments, the MFILE-DCS software may be configured to perform AI tool checks in the medical report such as AIDOC checks and Nanox checks.

In at least some embodiments, the MFILE-DCS software may be configured to check for CT exams with contrast and for subjects less than 3 years of age, and if so, certain text should be in report.

In at least some embodiments, the MFILE-DCS software may be configured to check for un-resolved problems in Medicalis, such as for example, any worklist tool and/or RIS worklist tool, for any accessions.

In at least some embodiments, the MFILE-DCS software may be configured to check if accession is still in a signed status.

In at least some embodiments, the MFILE-DCS software may be configured to check that Safe Sign was used when the medical report gets signed.

In at least some embodiments, the MFILE-DCS software may be configured to include a fail safe option to retract usage if server/network issues occur unexpectedly.

In at least some embodiments, the MFILE-DCS software may be configured to be applied to other systems like electronic medical records (EMR's) and other reports.

In at least some embodiments, the MFILE-DCS software may be configured to check if accession is still in a signed status.

In at least some embodiments, the MFILE-DCS software may be configured to check that Safe Sign was used when the medical report gets signed.

In at least some embodiments, the MFILE-DCS software may be configured to include a fail-safe option to retract usage if server/network issues occur unexpectedly.

In at least some embodiments, the MFILE-DCS software may be configured to check the start dates/privilege/credential dates to verify that the provider may provide care at the site of the report.

In at least some embodiments, the MFILE-DCS software may be configured to check if a subject is a Medicare (or other insurance provider) enrollee to verify that the signer has rights to sign report.

FIG. 8 is a screenshot 800 of an exemplary subject medical record with errors detected by the MFILE-DCS software in accordance with one or more embodiments of the present disclosure. In this case the MFILE-DCS software has identified a gender error 810 where the subject is flagged as being “female” but the medical report indicates that the prostate 820 (e.g., a male organ) appears normal.

FIG. 9 is a third summary diagram 900 of the MFILE-DCS software in accordance with one or more embodiments of the present disclosure. In a flow diagram 910, the healthcare provider may order an exam such as an X-ray to be performed on the subject. The radiologist may dictate the report and findings from the X-ray in a radiology report 930 and digitally signs the report 930. The MFILE-DCS software may analyze the radiology report as compared to other subject data and exams. The MFILE-DCS software may generate an MFILE-DCS output 920 after determining from the radiology report 930 the following errors: (1) an insufficient history of known liver cancer, (2) the AI may detect Liver Iron Overload, and (3) a left/right laterality error. In at least some embodiments, the MFILE-DCS software may then generate radiology report addendums 940 to correct the radiology report 930 using the medical report errors from the MFILE-DCS output 920.

In at least some embodiments, the corrections to the radiology report 930 may be performed manually. In other embodiments, the radiology report addendums 940 may be automatically generated by the MFILE-DCS software. In yet other embodiments, the corrections to the radiology report 930 may be performed before the radiologist safe signs the radiology report 930 to eliminate the need to generate the radiology report addendums 940.

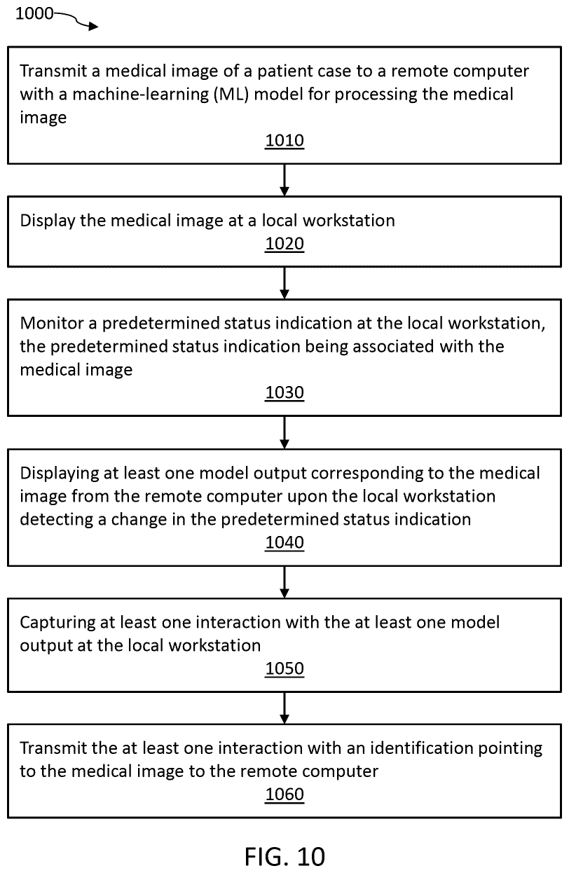

FIG. 10 is a flowchart illustrating an AI-enhanced radiologist's interpretation process 1000 in accordance with one or more embodiments of the present disclosure. The process 1000 is implemented with a computing system and includes exemplary blocks 1010-1060 as described hereinbelow.

In block 1010, the computing system transmits a medical image of a subject case to a remote computer with a machine-learning (ML) model for processing the medical image.

In block 1020, the computing system displays the medical image at a local workstation. In at least some embodiments, the local workstation on premises with a radiologist who generates or orders to generate the medical image. The computing system may belong to the hospital 130 the radiologist associated with. The remote computer is remote to the computing system.

In block 1030, the computing system monitors a predetermined status indication at the local workstation, the predetermined status indication being associated with the medical image. In at least some embodiments, the predetermined status indication represents whether the radiologist have entered report regarding the medical image on the local workstation.

In block 1040, the computing system displays at least one model output corresponding to the medical image from the remote computer upon the local workstation detecting a change in the predetermined status. In at least some embodiments, the change in the predetermined status indication represents that the radiologist has entered into the report regarding the medical image.

In block 1050, the computing system captures at least one interaction with the at least one model output at the local workstation. In at least some embodiments, the at least one interaction represents the radiologist's comments on the model output.

In block 1060, the computing system transmits the at least one interaction with an identification pointing to the medical image to the remote computer. As the at least one interaction represents the radiologist's comments on the model output, the remote computer can use the comments as training data to retrain the ML model.

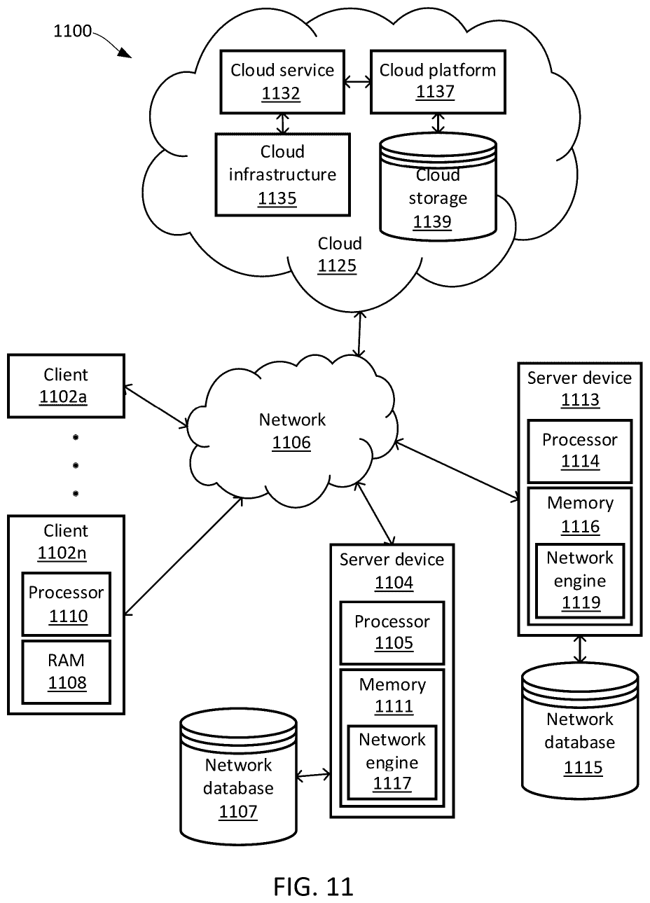

FIG. 11 is a block diagram illustrating an exemplary computer-based system/platform 1100 in accordance with one or more embodiments of the present disclosure. However, not all of these components may be required to practice one or more embodiments, and variations in the arrangement and type of the components may be made without departing from the spirit or scope of various embodiments of the present disclosure. In at least some embodiments, client computing devices 1102a through 1102n shown each at least includes a computer-readable medium, such as a random-access memory (RAM) 1108 coupled to a processor 1110 or FLASH memory (not shown) associated thereto. In at least some embodiments, processor 1110 may execute computer-executable program instructions stored in memory 1108. In at least some embodiments, processor 1110 may include a microprocessor, an ASIC, and/or a state machine. In at least some embodiments, processor 1110 may include, or may be in communication with, media, for example computer-readable media, which stores instructions that, when executed by processor 1110, may cause processor 1110 to perform one or more steps described herein. In at least some embodiments, examples of computer-readable media may include, but are not limited to, an electronic, optical, magnetic, or other storage or transmission device capable of providing a processor, such as the processor 1110 of client 1102a, with computer-readable instructions. In at least some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In at least some embodiments, the instructions may comprise code from any computer-programming language, including, for example, C, C++, Visual Basic, Java, Python, Perl, JavaScript, etc.

In at least some embodiments, client computing devices 1102a through 1102n may also comprise a number of external or internal devices such as a mouse, a CD-ROM, DVD, a physical or virtual keyboard, a display, a speaker, or other input or output devices. In at least some embodiments, examples of client computing devices 1102a through 1102n (e.g., clients) may be any type of processor-based platforms that are connected to a network 1106 such as, without limitation, personal computers, digital assistants, personal digital assistants, smart phones, pagers, digital tablets, laptop computers, Internet appliances, and other processor-based devices. In at least some embodiments, client computing devices 1102a through 1102n may be specifically programmed with one or more application programs in accordance with one or more principles/methodologies detailed herein. In at least some embodiments, client computing devices 1102a through 1102n may operate on any operating system capable of supporting a browser or browser-enabled application, such as Microsoft™, Windows™, and/or Linux. In at least some embodiments, client computing devices 1102a through 1102n shown may include, for example, personal computers executing a browser application program such as Microsoft Corporation's Internet Explorer™, Apple Computer, Inc.'s Safari™, Mozilla Firefox, and/or Opera. In at least some embodiments, through the client computing client devices 1102a through 1102n, users may communicate over exemplary network 1106 with each other and/or with other systems and/or devices coupled to network 1106.

As shown in FIG. 11, exemplary server devices 1104 and 1113 may be also coupled to network 506. Exemplary server device 1104 may include a processor 1105 coupled to a memory 1111 that may store a network engine 1117. Exemplary server device 1113 may include a processor 1114 coupled to a memory 1116 that may store a network engine 1119. In at least some embodiments, one or more client computing devices 1102a through 1102n may be mobile clients.

As shown in FIG. 11, exemplary server device 1104 may be coupled to an exemplary network database 1107; and exemplary server device 1113 may be coupled to an exemplary network database 1115. In at least some embodiments, exemplary databases 1107 and 1115 may be any type of database, including a database managed by a database management system (DBMS). In at least some embodiments, an exemplary DBMS-managed database may be specifically programmed as an engine that controls organization, storage, management, and/or retrieval of data in the respective database. In at least some embodiments, the exemplary DBMS-managed database may be specifically programmed to provide the ability to query, backup and replicate, enforce rules, provide security, compute, perform change and access logging, and/or automate optimization. In at least some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In at least some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In at least some embodiments, the exemplary DBMS-managed database may be specifically programmed to include metadata about the data that is stored.

As shown in FIG. 11, network 1106 may be coupled to a cloud computing/architecture(s) 1125. Cloud computing/architecture(s) 1125 may include a cloud service 1132 coupled to a cloud infrastructure 1135 and a cloud platform 1137, where the cloud platform 1137 may be coupled to a cloud storage 1139.

The computing system can also employ any number of software, firmware, and/or hardware configurations. For example, one or more of the example embodiments disclosed herein can be encoded as a computer program (also referred to as computer software, software applications, computer-readable instructions, and/or computer control logic) on a computer-readable medium.

The term “computer-readable medium,” as used herein, can generally refer to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In at least some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment may be implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In at least some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

In at least some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) FreeBSD, NetBSD, OpenBSD; (2) Linux; (3) Microsoft Windows; (4) OS X (MacOS); (5) MacOS 11; (6) Solaris; (7) Android; (8) iOS; (9) Embedded Linux; (10) Tizen; (11) WebOS; (12) IBM i; (13) IBM AIX; (14) Binary Runtime Environment for Wireless (BREW); (15) Cocoa (API); (16) Cocoa Touch; (17) Java Platforms; (18) JavaFX; (19) JavaFX Mobile; (20) Microsoft DirectX; (21) . NET Framework; (22) Silverlight; (23) Open Web Platform; (24) Oracle Database; (25) Qt; (26) Eclipse Rich Client Platform; (27) SAP NetWeaver; (28) Smartface; and/or (29) Windows Runtime.

In at least some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

As used herein, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In at least some embodiments, the exemplary inventive computer-based systems/platforms, the exemplary inventive computer-based devices, and/or the exemplary inventive computer-based components of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

The aforementioned examples are, of course, illustrative, and not restrictive.

As used herein, the term “user” shall have a meaning of at least one user. In at least some embodiments, the terms “user”, “subscriber” “consumer” or “customer” should be understood to refer to a user of an application or applications for implementing the functions of the CVCP as described herein and/or a consumer of data supplied by a data provider. By way of example, and not limitation, the terms “user” or “subscriber” can refer to a person who receives data provided by the data or service provider over the Internet in a browser session or can refer to an automated software application which receives the data and stores or processes the data.

The aforementioned examples are, of course, illustrative, and not restrictive.

In at least some embodiments, the exemplary inventive computer-based systems, the exemplary inventive computer-based devices, and/or the exemplary inventive computer-based components of the present disclosure may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In at least some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In at least some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model incrementally,
iv) determine the accuracy for a specific number of timesteps,
v) apply the exemplary trained model to process the newly received input data, vi) optionally and in parallel, continue to train the exemplary trained model with a predetermined periodicity.

In at least some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In at least some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node may be activated. In at least some embodiments and, optionally, in combination of any embodiment described above or below, the exemplary aggregation function may be a mathematical function that combines (e.g., sum, product, etc.) input signals to the node. In at least some embodiments and, optionally, in combination of any embodiment described above or below, an output of the exemplary aggregation function may be used as input to the exemplary activation function. In at least some embodiments and, optionally, in combination of any embodiment described above or below, the bias may be a constant value or function that may be used by the aggregation function and/or the activation function to make the node more or less likely to be activated.

The aforementioned examples are, of course, illustrative, and not restrictive.

At least some aspects of the present disclosure will now be described with reference to the following numbered clauses.

Clause 1. A computer-implemented method including: transmitting, by at least one computing device, a first medical image of a first subject case to a first remote computer with a first machine-learning (ML) model for processing the first medical image; displaying, by the at least one computing device, the first medical image at a local workstation; monitoring, by the at least one computing device, a predetermined status indication at the local workstation, the predetermined status indication being associated with the first medical image; displaying, by the at least one computing device, at least one first model output corresponding to the first medical image from the first remote computer upon the local workstation detecting a change in the predetermined status indication; capturing, by the at least one computing device, at least one first interaction with the at least one first model output at the local workstation; and transmitting, by the at least one computing device, the at least one first interaction with an identification pointing to the first medical image to the first remote computer.

Clause 2. The method of clause 1, further including displaying, by the at least one computing device, a text box at the local workstation for entering a text, wherein the change in the status indication is triggered by a number of characters entered in the text box exceeding a predetermined threshold.

Clause 3. The method of clause 2, further including displaying, by the at least one computing device, a button at the local workstation, wherein a clicking of the button triggers a change of the status indication.

Clause 4. The method of clause 3, wherein the button switches from invisible to visible upon a text curser being placed in the text box.

Clause 5. The method of clause 1, wherein the change in the predetermined status indication corresponds to completion of a diagnostic conclusion section of a radiology report.

Clause 6. The method of clause 1, further including: transmitting, by the at least one computing device, a second medical image of a second subject case to the first remote computer with the first machine-learning model for processing the second medical image; receiving, by the at least one computing device, an indication that the second subject case has lower priority ranking than the first subject case before displaying the first medical image at the local workstation.

Clause 7. The method of clause 1, further including incrementing, by the at least one computing device, a counter by 1 in response to transmitting the at least one first interaction to the first remote computer.

Clause 8. The method of clause 1, wherein the first remote computer retrains the first ML model with the captured at least one first interaction.

Clause 9. The method of clause 1, further including: transmitting, by the at least one computing device, the first medical image to a second remote computer with a second ML model for processing the first medical image; receiving, by the at least one computing device, at least one second model output corresponding to the first medical image from the second remote computer; and displaying, by the at least one computing device, the at least one second model output along with the at least one first model output.

Clause 10. The method of clause 9, further including normalizing the at least one first and second model output into a predetermined schema including at least one of: standardized labels, confidence scores, anatomical locations, or clinical categories.

Clause 11. The method of clause 9, further including comparing the at least one first model output with the at least one second model output to identify agreement, discrepancy, or conflict therebetween.

Clause 12. The method of clause 1, further including displaying a user-selectable control that allows a radiologist to manually retrieve the at least one first model output after the local workstation has detected the change in the predetermined status indication.

Clause 13. The method of clause 1, wherein at least one first interaction includes an accept, reject, or modify action applied to the at least one first model output.

Clause 14. The method of clause 1, further including anonymizing, by the computing device, the at least one first interaction before transmitting the at least one first interaction to the first remote computer.

Clause 15. The method of clause 1, further including providing, by the at least one computing device, an integrated procedure complication reporting interface configured to capture structured data identifying a procedural complication type, a complication category, and associated descriptive information during or after interpretation of the first medical image.

Clause 16. The method of clause 15, wherein the integrated procedure complication reporting interface includes a validation logic adapted to prevent submission of a complication report unless a predetermined field has been completed.

Clause 17. The method of clause 1, further including automatically categorizing, by the at least one computing device, the at least one first model output within a clinical report into a predefined category including at least one of critical, urgent, or incidental findings, and updating a navigation interface based on the categorization, wherein the navigation interface is configured to allow a user to selectively access categorized findings and corresponding follow-up actions without manual tracking.

Clause 18. The method of clause 1, further including detecting, by the at least one computing device, a duplicate subject identifier associated with the first medical image and presenting, by the at least one computing device, at least one prior imaging study associated with the duplicate identifier at the local workstation.

Clause 19. A non-transitory computer readable medium storing program instructions that, when executed by a processor, cause a computer system to perform operations including: transmitting a first medical image of a first subject case to a first remote computer with a first machine-learning (ML) model for processing the first medical image; displaying the first medical image at a local workstation; monitoring a predetermined status indication at the local workstation, the predetermined status indication being associated with the first medical image; displaying at least one first model output corresponding to the first medical image from the first remote computer upon the local workstation detecting a change in the predetermined status indication; capturing at least one first interaction with the at least one first model output at the local workstation; and transmitting the at least one first interaction with an identification pointing to the first medical image to the first remote computer.

Clause 20. A system, including: at least one processor; and a memory in communication with the at least one processor and storing instructions that, when executed by the at least one processor, cause the at least one processor to: transmit a first medical image of a first subject case to a first remote computer with a first machine-learning (ML) model for processing the first medical image; display the first medical image at a local workstation; monitor a predetermined status indication at the local workstation, the predetermined status indication being associated with the first medical image; display at least one first model output corresponding to the first medical image from the first remote computer upon the local workstation detecting a change in the predetermined status indication; capture at least one first interaction with the at least one first model output at the local workstation; and transmit the at least one first interaction with an identification pointing to the first medical image to the first remote computer.

Publications cited throughout this document are hereby incorporated by reference in their entirety. While one or more embodiments of the present disclosure have been described, it may be understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A computer-implemented method comprising:
transmitting, by at least one computing device, a first medical image of a first subject case to a first remote computer with a first machine-learning (ML) model for processing the first medical image;
displaying, by the at least one computing device, the first medical image at a local workstation;
monitoring, by the at least one computing device, a predetermined status indication at the local workstation, the predetermined status indication being associated with the first medical image;
displaying, by the at least one computing device, at least one first model output corresponding to the first medical image from the first remote computer upon the local workstation detecting a change in the predetermined status indication;
capturing, by the at least one computing device, at least one first interaction with the at least one first model output at the local workstation; and
transmitting, by the at least one computing device, the at least one first interaction with an identification pointing to the first medical image to the first remote computer.

2. The method of claim 1, further comprising displaying, by the at least one computing device, a text box at the local workstation for entering a text, wherein the change in the status indication is triggered by a number of characters entered in the text box exceeding a predetermined threshold.

3. The method of claim 2, further comprising displaying, by the at least one computing device, a button at the local workstation, wherein a clicking of the button triggers a change of the status indication.

4. The method of claim 3, wherein the button switches from invisible to visible upon a text curser being placed in the text box.

5. The method of claim 1, wherein the change in the predetermined status indication corresponds to completion of a diagnostic conclusion section of a radiology report.

6. The method of claim 1, further comprising:
transmitting, by the at least one computing device, a second medical image of a second subject case to the first remote computer with the first machine-learning model for processing the second medical image;
receiving, by the at least one computing device, an indication that the second subject case has lower priority ranking than the first subject case before displaying the first medical image at the local workstation.

7. The method of claim 1, further comprising incrementing, by the at least one computing device, a counter by 1 in response to transmitting the at least one first interaction to the first remote computer.

8. The method of claim 1, wherein the first remote computer retrains the first ML model with the captured at least one first interaction.

9. The method of claim 1, further comprising:
transmitting, by the at least one computing device, the first medical image to a second remote computer with a second ML model for processing the first medical image;
receiving, by the at least one computing device, at least one second model output corresponding to the first medical image from the second remote computer; and
displaying, by the at least one computing device, the at least one second model output along with the at least one first model output.

10. The method of claim 9, further comprising normalizing the at least one first and second model output into a predetermined schema comprising at least one of: standardized labels, confidence scores, anatomical locations, or clinical categories.

11. The method of claim 9, further comprising comparing the at least one first model output with the at least one second model output to identify agreement, discrepancy, or conflict therebetween.

12. The method of claim 1, further comprising displaying a user-selectable control that allows a radiologist to manually retrieve the at least one first model output after the local workstation has detected the change in the predetermined status indication.

13. The method of claim 1, wherein at least one first interaction comprises an accept, reject, or modify action applied to the at least one first model output.

14. The method of claim 1, further comprising anonymizing, by the computing device, the at least one first interaction before transmitting the at least one first interaction to the first remote computer.

15. The method of claim 1, further comprising providing, by the at least one computing device, an integrated procedure complication reporting interface configured to capture structured data identifying a procedural complication type, a complication category, and associated descriptive information during or after interpretation of the first medical image.

16. The method of claim 15, wherein the integrated procedure complication reporting interface comprises a validation logic adapted to prevent submission of a complication report unless a predetermined field has been completed.

17. The method of claim 1, further comprising automatically categorizing, by the at least one computing device, the at least one first model output within a clinical report into a predefined category comprising at least one of critical, urgent, or incidental findings, and updating a navigation interface based on the categorization, wherein the navigation interface is configured to allow a user to selectively access categorized findings and corresponding follow-up actions without manual tracking.

18. The method of claim 1, further comprising detecting, by the at least one computing device, a duplicate subject identifier associated with the first medical image and presenting, by the at least one computing device, at least one prior imaging study associated with the duplicate identifier at the local workstation.

19. A non-transitory computer readable medium storing program instructions that, when executed by a processor, cause a computer system to perform operations comprising:
transmitting a first medical image of a first subject case to a first remote computer with a first machine-learning (ML) model for processing the first medical image;
displaying the first medical image at a local workstation;
monitoring a predetermined status indication at the local workstation, the predetermined status indication being associated with the first medical image;
displaying at least one first model output corresponding to the first medical image from the first remote computer upon the local workstation detecting a change in the predetermined status indication;
capturing at least one first interaction with the at least one first model output at the local workstation; and
transmitting the at least one first interaction with an identification pointing to the first medical image to the first remote computer.

20. A system, comprising:
at least one processor; and
a memory in communication with the at least one processor and storing instructions that, when executed by the at least one processor, cause the at least one processor to:
transmit a first medical image of a first subject case to a first remote computer with a first machine-learning (ML) model for processing the first medical image;
display the first medical image at a local workstation;
monitor a predetermined status indication at the local workstation, the predetermined status indication being associated with the first medical image;
display at least one first model output corresponding to the first medical image from the first remote computer upon the local workstation detecting a change in the predetermined status indication;
capture at least one first interaction with the at least one first model output at the local workstation; and
transmit the at least one first interaction with an identification pointing to the first medical image to the first remote computer.
